(19) United States
(12) Patent Application Publication
(10) Pub. No.: US 2024/0132660 A1
(43) Pub. Date: Apr. 25, 2024

(54) COMPOSITION COMPRISING ACTIVATED AND FUNCTIONALIZED PRE-POLYMER

(71) Applicant: TISSIUM SA, Paris (FR)

(72) Inventors: Camille LEGROS, Paris (FR); Benoît RHONE, Mantes la Jolie (FR)

(21) Appl. No.: 18/546,198

(22) PCT Filed: Feb. 22, 2022
(86) PCT No.: PCT/EP2022/054401

§ 371 Date: Aug. 11, 2023

(30) Foreign Application Priority Data
Feb. 24, 2021 (EP) 21315027.9

(51) Int. Cl. C08G 63/91; A61L 24/06; C09J 167/02

(52) U.S. Cl. CPC C08G 63/914; A61L 24/06; C09J 167/02; A61L 2300/802

(57) ABSTRACT

The present disclosure relates to a composition comprising: a pre-polymer having activated groups and negatively-charged functional groups on a polymeric backbone. The present disclosure also relates to a method for preparing such a composition.

### FIELD OF INVENTION

The present invention relates to a composition comprising an activated and functionalized pre-polymer, a method of manufacturing the composition, a method of curing the composition, a cured composition obtainable therefrom, uses of the composition and methods of using the composition.

### BACKGROUND OF THE INVENTION

Open heart surgery typically relies on a suture-based closure or attachment of cardiovascular structures. However, this can be technically challenging due to the fragility of young infant tissue and diseased or damaged adult tissue, leading to longer operative times, increased risk of complications of bleeding or dehiscence, and therefore worse outcomes. Furthermore, cardiopulmonary bypass (CPB) is required for open heart surgery, and this has significant adverse effects, including an inflammatory response and potential neurological complications.

While catheter-based interventions for closure of cardiac defects such as atrial and ventricular septal defects (ASDs and VSDs) have recently emerged in an effort to reduce the invasiveness of the procedures, major challenges remain with securing devices inside the beating heart. Specifically, fixation of devices for catheter-based closure of cardiac septal defects currently relies on mechanical means of gripping tissue. This can cause injury to critical structures, such as heart valves or specialized conduction tissue. Furthermore, if inadequate tissue rims exist around defects, the prosthesis may dislodge, damaging the neighboring structures and also leaving residual defects, limiting device application. Therefore, such methods can only be applied in select patients, depending on the anatomic location and the geometric shape of the defect.

Soft and compliant tissue adhesives that cure rapidly could be used to attach tissue surfaces together or prosthetic devices to tissue without the need for mechanical entrapment or fixation, thereby avoiding tissue compression and erosion. Such materials could find a broad range of applications not only in minimally invasive cardiac repair, but also in the repair of soft tissues potentially with minimal scarring and damage. For example, in vascular surgery, suture-based anastomosis does not always result in an instantaneous hemostatic seal and can create irregularities in the endothelium that predispose to thrombosis. Furthermore, the presence of permanent sutures can cause a foreign body reaction with further inflammation and scarring at the repair site, which may increase the risk of late vessel occlusion. Tissue adhesives could accomplish such repairs with an instantaneous seal and with minimal scarring or tissue damage.

Current clinically-available adhesives, such as medical grade cyanoacrylate (CA) or fibrin sealant, are easily washed out or cured under dynamic wet conditions, are toxic and cannot be used internally, and/or exhibit weak adhesive properties such that they cannot withstand the forces inside the cardiac chambers and major blood vessels. Also, many of these adhesives exhibit activation properties that make fine adjustments or repositioning of the devices very difficult. Moreover, many adhesives under development achieve tissue adhesion only through chemical reaction with functional groups at the tissue surface, and thus become ineffective in the presence of blood.

Alternatives to cyanoacrylate have been explored. U.S. Pat. No. 8,143,042 B2 describes biodegradable elastomers prepared by crosslinking a prepolymer containing crosslinkable functional groups, such as acrylate groups. It also discloses that it is desirable to increase the number of free hydroxyl groups on the polymer in order to increase the stickiness of the polymer. Increasing the number of hydroxyl groups in the backbone also leads to enhanced solubility in physiologic solutions. This suggests that the primary mechanism of adhesion of the polymer is chemical interactions between functional groups, for example free hydroxyl groups on the polymer and the tissue to which it is applied. However, this type of chemical interaction becomes ineffective in the presence of body fluids, especially blood, as shown in Artzi et al., Adv. Mater. 21, 3399-3403 (2009).

Similarly, Mandavi et al., 2008, PNAS, 2307-2312, describes nanopatterned elastomeric polymer and proposes applying a thin layer of oxidized dextran with aldehyde functionalities (DXTA) to increase adhesion strength of the adhesive by promoting covalent cross-linking between terminal aldehyde group in DXTA with amine groups in proteins of tissue. This adhesion mechanism based essentially on covalent bonding between the radicals generated during the curing process and functional groups of the tissue has several limitations. The use of adhesives with reactive chemistry requires tissue surfaces to be dried prior to application of the pre-polymer, which makes it very challenging to use in cardiac application, such as during emergency procedures. Additionally, reactive chemistry can denature proteins or tissue and promote undesirable immune reaction such as local inflammation which can lead to adhesive rejection. Moreover, reactive chemistry that only bonds to the surface of tissue would likely have lower adhesion as the interface would be more distinct, and thus there would be a mismatch in mechanical properties at the interface between the glue and tissue.

Elastomeric crosslinked polyesters are disclosed in US 2013/0231412 A1. Biodegradable polymers are disclosed in U.S. Pat. No. 7,722,894 B2. Adhesive articles are disclosed in WO2009/067482 A1 and WO2014/190302 A1. Blood resistant surgical glue is described in Lang et al. “A Blood-Resistant Surgical Glue for Minimally Invasive Repair of Vessels and Heart Defects” Sci Transl Med 8 Jan. 2014: Vol. 6, Issue 218, p. 218ra6 and WO2014/190302 A1.

A phosphate functionalized biodegradable polymer for bone tissue engineering, phosphorylated poly(sebacoyl diglyceride), is disclosed by Huang, P. et al. in J. Mater. Chem. B 4, 2090-2101 (2016). This polymer was designed for use in bone regeneration; phosphate groups are incorporated for their osteo-inductive properties.

### SUMMARY OF THE INVENTION

The invention provides an improved and commercially viable activated and functionalized pre-polymer that can be readily applied to the desired site, is biocompatible (non-toxic), and exhibits strong adhesive forces once cured/crosslinked leading to improved tissue sealant/adhesive.

The improved activated and functionalized pre-polymer remains in place at the desired site prior to curing/crosslinking, even in the presence of bodily fluids, such as blood.

The improved activated and functionalized pre-polymer is stable when stored.

More particularly, the invention provides a pre-polymer having activated groups and negatively-charged functional groups on a polymeric backbone, wherein the proportion of negatively-charged functional groups compared to the number of monomer units in the backbone is at least 0.05 mol/mol of monomer unit (e.g., 0.2 mol/mol of monomer unit).

The present invention also provides a method for preparing the composition of the present invention.

The present invention further provides a method of curing the composition according to the present invention, comprising curing the composition with a stimulus, for example light in the presence of a photo-initiator.

The present invention also provides a cured composition obtainable by the curing method according to the present invention. Said cured composition is desirably an adhesive, i.e., one that can bind strongly to a surface or can bind one surface to another.

The present invention further provides methods of use and use of the composition according to the present invention for gluing or sealing tissue or for adhering a medical device to the surface of tissue.

The inventors have found that, compared to known compositions, the present invention offers advantages which are not found in the prior art.

### BRIEF DESCRIPTION OF THE FIGURES

FIG. 1 shows a synthetic route to a composition according to the invention.

FIG. 2 shows a synthetic route to another composition according to the invention.

FIG. 3 shows a synthetic route to another composition according to the invention.

FIG. 4 shows a synthetic route to another composition according to the invention.

### DETAILED DESCRIPTION OF THE INVENTION

### Pre-Polymer

Preferably, the polymeric backbone of the pre-polymer comprises a polymeric unit of the general formula (—A—B—)ₙ, wherein A is derived from a substituted or unsubstituted polyol or mixture thereof, and B is derived from a substituted or unsubstituted polyacid or mixture thereof; and n represents an integer greater than 1. The polymeric backbone is made up of repeating monomer units of general formula —A—B.

The term “substituted” has its usual meaning in chemical nomenclature and is used to describe a chemical compound in which a hydrogen on the primary carbon chain has been replaced with a substituent such as alkyl, aryl, carboxylic acid, ester, amide, amine, urethane, ether, or carbonyl.

Component A of the pre-polymer may be derived from a polyol or mixture thereof, such as a diol, triol, tetraol or higher polyols. Suitable polyols include diols, such as alkane diols, preferably octanediol; triols, such as glycerol, trimethylolpropane, trimethylolpropane ethoxylate, triethanolamine; tetraols, such as erythritol, pentaerythritol; and higher polyols, such as sorbitol. Component A may also be derived from unsaturated polyols, such as tetradeca-2,12-diene-1,14-diol, polybutadiene-diol or other polyols including macromonomer polyols such as, for example polyethylene oxide, polycaprolactone triol and N-methyldiethanoamine (MDEA) can also be used. Preferably, the polyol is substituted or unsubstituted glycerol.

Component B of the pre-polymer is derived from a polyacid or mixture thereof, preferably diacid or triacid. Exemplary acids include, but are not limited to, glutaric acid (5 carbons), adipic acid (6 carbons), pimelic acid (7 carbons), sebacic acid (8 carbons), azelaic acid (9 carbons) and citric acid. Exemplary long chain diacids include diacids having more than 10, more than 15, more than 20, and more than 25 carbon atoms. Non-aliphatic diacids can also be used. For example, versions of the above diacids having one or more double bonds can be used to produce polyol-diacid co-polymers. Preferably the polyacid is substituted or unsubstituted sebacic acid.

Polyol-based polymers described in US 2011/0008277, U.S. Pat. Nos. 7,722,894 and 8,143,042, the contents of which are hereby incorporated by reference, are suitable polymeric backbones for use in the present invention.

Several substituents, such as amines, aldehydes, hydrazides, acrylates and aromatic groups, can be incorporated into the carbon chain. Exemplary aromatic diacids include terephthalic acid and carboxyphenoxy-propane. The diacids can also include substituents. For example, reactive groups like amine and hydroxy can be used to increase the number of sites available for cross-linking. Amino acids and other biomolecules can be used to modify the biological properties. Aromatic groups, aliphatic groups, and halogen atoms can be used to modify the inter-chain interactions within the polymer.

Alternatively, the polymeric backbone of the pre-polymer is a polyamide or polyurethane backbone. For example, polyamine (comprising two or more amino groups) may be used to react with polyacid together with polyol or after reacting with polyol. Exemplary poly(ester amide) includes those described in Cheng et al., Adv. Mater. 2011, 23, 1195-11100, the contents of which are herein incorporated by reference. In other examples, polyisocyanates (comprising two or more isocyanate groups) may be used to react with polyacid together with polyol or after reacting with polyol. Exemplary polyester urethanes include those described in US 2013/231412.

The weight average molecular weight of the pre-polymer (Mw), measured by Gel Permeation Chromatography equipped with a refractive index, may be from about 1,000 Daltons to about 1,000,000 Daltons, preferably from about 2,000 Daltons to about 500,000 Daltons, more preferably from about 2,000 Daltons to about 250,000 Daltons, most preferably from about 2,000 Daltons to about 100,000 Daltons. The weight average molecular weight may be less than about 100,000 Dalton, less than about 75,000 Daltons, less than about 50,000 Daltons, less than about 40,000 Daltons, less than about 30,000 Daltons, or less than about 20,000 Daltons. The weight average molecular weight may be from about 1,000 Daltons to about 10,000 Daltons, from about 2,000 Daltons to about 10,000 Daltons, from about 3,000 Daltons to about 10,000 Daltons, from about 5,000 Daltons to about 10,000 Daltons. Preferably, it is about 4,500 Daltons.

The term “about” as used herein means within 10%, preferably within 8%, and more preferably within 5% of a given value or range. According to a specific embodiment, “about X” means X, when X refers to the value or range.

The pre-polymer may have a polydispersity, measured by Gel Permeation Chromatography equipped with a refractive index, below 20.0, more preferably below 10.0, more preferably below 5.0, and even more preferably below 2.5. Preferably, it is about 2.5.

The molar ratios of the polyol to the polyacid in the pre-polymer are suitably in the range of about 0.5:1 to about 1.5:1, preferably in the range of about 0.9:1.1 to about 1.1:0.9 and most preferably about 1:1.

### Activated Pre-Polymer

The pre-polymer in the composition of the invention has activated groups on its polymeric backbone.

The activated groups are functional groups that can react or be reacted to form crosslinks. The pre-polymer is activated by reacting one or more functional groups on the monomer units of the backbone to provide one or more functional groups that can react or be reacted to form crosslinks resulting in cured polymer. According to an embodiment, the pre-polymer has activated groups of different nature on its backbone monomeric units. The polymeric backbone of the pre-polymer may comprise a polymeric unit of the general formula (—A—B—)ₙ, wherein A is derived from a substituted or unsubstituted polyol or mixture thereof, and B is derived from a substituted or unsubstituted polyacid or mixture thereof.

Suitable functional groups to be activated on the pre-polymer backbone include hydroxy groups, carboxyl groups, amines, and combinations thereof, preferably hydroxy and/or carboxyl groups. The free hydroxyl or carboxylic acid groups on the pre-polymer can be activated by functionalizing the hydroxy groups with a moiety which can form a crosslink between polymer chains. The groups that are activated can be free hydroxyl or carboxylic acid groups on A and/or B moieties in the pre-polymer.

The free hydroxy or carboxyl groups can be functionalized with a variety of functional groups, for example vinyl groups. Vinyl groups can be introduced by a variety of techniques known in the art, such as by vinylation or acrylation. According to the present invention, vinyl groups contain the following structure —CRₓ═CR_{y}R_{z} wherein Rₓ, R_{y}, R_{z} are independently from one another, selected from the group consisting of H, alkyl, such as methyl or ethyl, aryl, such as phenyl, substituted alkyl, substituted aryl, carboxylic acid, ester, amide, amine, urethane, ether, and carbonyl.

Preferably, the activated group is or contains an acrylate group. According to the present invention, acrylate groups may contain the following group: —C(═O)—CRₚ═CR_{q}Rᵣ, wherein Rₚ, R_{q}, Rᵣ are independently from one another, selected from the group consisting of H, alkyl, such as methyl or ethyl, aryl, such as phenyl, substituted alkyl, substituted aryl, carboxylic acid, ester, amide, amine, urethane, ether, and carbonyl. According to an embodiment, the activated pre-polymer contains a mixture of different acrylate groups. According to an embodiment, the activated pre-polymer contains methacrylate group.

Preferably, all or part of the acrylate groups containing the —C(═O)—CRₚ═CR_{q}Rᵣ group are such that Rₚ, R_{q} and Rᵣ are H; or such that Rₚ is CH₃, R_{q} and Rᵣ are H; or such that Rₚ and R_{q} are H and Rᵣ is CH₃; or such that Rₚ and R_{q} are H and Rᵣ is phenyl.

Vinyl groups can also be incorporated in the backbone of the pre-polymer using free carboxyl groups on the pre-polymer. For example, hydroxyethyl methacrylate can be incorporated through the COOH groups of the pre-polymer using carbonyl diimidazole activation chemistry.

In an embodiment of the invention, at least a proportion of the activated groups on the polymeric backbone of the pre-polymer may be alkene groups (e.g., acrylate, methacrylate). The degree of activation (e.g., acrylation) is suitably measured by a technique such as ¹H NMR. The degree of activation (e.g., acrylation) is suitably characterized as “DA”. The proportion of activated groups may be compared to the number of monomer units in the backbone. This can vary and can be from 0.1 to 0.8 mol/mol of monomer unit, preferably from 0.2 to 0.6 mol/mol of monomer unit and most preferably from 0.3 to 0.45 mol/mol of monomer unit, such as 0.3 mol/mol of monomer unit, for achieving optimal adhesive or burst performance properties at room temperature or elevated temperature up to 40° C., preferably 37° C. It is most preferred when the degree of activation is as described above and the reactive functional group is acrylate (e.g. methacrylate) i.e., degree of acrylation as above. When the polymeric unit of the backbone is of the general formula (—A—B—)ₙ, with A derived from a substituted or unsubstituted polyol and B derived from a substituted or unsubstituted polyacid, the monomer unit is of general formula —A—B— and the proportion of activated groups may be quoted per mole of polyacid or per mole of polyol. The DA ranges quoted above are preferably mol/mol of polyacid.

The pre-polymer in the composition of the invention is preferably derived from an activated pre-polymer that has the general formula (I):

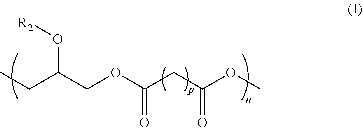

wherein n and p each independently represent an integer equal to or greater than 1, and wherein R₂ in each individual unit represents hydrogen or a polymer chain or —C(═O)—CR₃═CR₄R₅, or C(═O)NR₆—CR₇R₈—CR₉R₁₀—O—C(═O)—CR₃═CR₄R₅, wherein R₃, R₄, R₅, R₆, R₇, R₈, R₉ and R₁₀ are independently from one another, selected from the group consisting of H, alkyl, such as methyl or ethyl, aryl, such as phenyl, substituted alkyl, substituted aryl, carboxylic acid, ester , amide, amine, urethane, ether, and carbonyl.

Preferably, R₃, R₄ and R₅ are H; or R₃ is CH₃, R₄ and R₅ are H; or R₃ and R₄ are H and R₅ is CH₃; or R₃ and R₄ are H and R₅ is phenyl. Preferably R₆, R₇, R₈, R₉ and R₁₀ are H.

Preferably, p is an integer from 1-20, more preferably from 2-10, even more preferably from 4-10. It is most preferred when p=8.

Preferably, the pre-polymer in the composition of the invention is derived from an activated pre-polymer that has a monomer unit of the general formula (II):

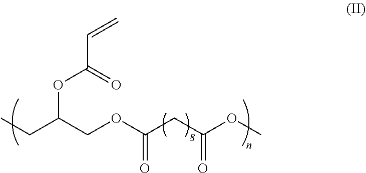

wherein n represents an integer equal to or greater than 1.

Preferably, the pre-polymer is derived from monomer units of general formula (II), e.g. 10% to 80% of the polymer backbone is derived from monomer units of general formula (II), preferably from 20% to 60% and most preferably from 30% to 45%.

In addition to acrylates or other vinyl groups, other agents can be used to provide activated groups on the pre-polymer backbone. Examples of such agents include, but are not limited to, glycidyl, epichlorohydrin, triphenylphosphine, diethyl azodicarboxylate (DEAD), diazirine, divinyladipate, and divinylsebacate with the use of enzymes as catalysts, phosgene-type reagents, di-acid chlorides, bis-anhydrides, bis-halides, metal surfaces, and combinations thereof. Agents may further include isocyanate, aldehyde, epoxy, vinyl ether, thiol, DOPA residues or N-Hydroxysuccinimide functional groups.

### Negatively-Charged Groups

The pre-polymer in the composition of the invention has negatively-charged functional groups on its polymeric backbone.

A negatively-charged functional group is a functional group that has a non-transient negative charge. When in aqueous solution, many negatively-charged groups are in equilibrium with their neutral counterpart. However, the negatively-charged functional groups of the present invention are usually present in the negatively-charged form and are only transiently present in the neutral form. The equilibrium between the negatively-charged and neutral forms will be affected by conditions such as pH, temperature and pressure. The negatively-charged functional groups of the present invention are predominantly present in the negatively-charged form at neutral pH (pH 7) and at room temperature and pressure; they are only transiently present in the neutral form.

The negatively-charged groups may include oxygen atoms. Suitable negatively-charged groups include phosphate groups (e.g., —O—P(OH)O₂⁻ and —O—PO₃²⁻), sulphate groups (e.g., —O—SO₃⁻) and carboxylate groups.

In an embodiment, at least a proportion of monomer repeating units on the polymeric backbone of the pre-polymer may already include a negatively-charged functional group. For example, there may be carboxylate groups (i.e., —COO—) groups on the polymeric backbone, including at the terminal ends of the polymeric backbone.

In another embodiment of the invention, at least a proportion of the activated groups (e.g., acrylate) on the polymeric backbone of the pre-polymer have reacted with a compound containing a negatively-charged or chargeable atom.

In the composition according to the present invention, the proportion of negatively-charged functional groups compared to the number of monomer units in the backbone is at least 0.05 mol/mol of monomer unit. Preferably the proportion is at least 0.1 mol/mol of monomer unit, more preferably at least 0.2 mol/mol of monomer unit. The proportion of negatively-charged groups is suitably measured by a technique such as ¹H NMR. When the polymeric unit of the backbone is of the general formula (—A—B—)ₙ, with A derived from a substituted or unsubstituted polyol and B derived from a substituted or unsubstituted polyacid, the monomer unit is of general formula —A—B— and the proportion of negatively-charged functional groups may be quoted per mole of polyacid or per mole of polyol. The ranges quoted above are preferably mol/mol of polyacid.

In an embodiment of the invention the pre-polymer is of formula (III):

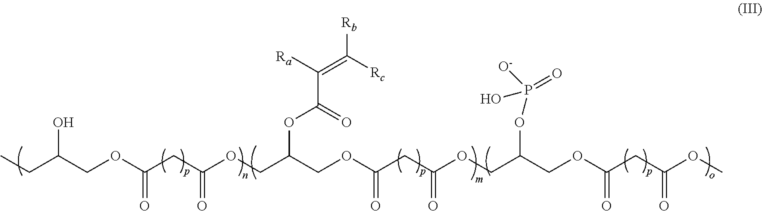

wherein p is an integer between 1 and 20, wherein n, m and o are integers equal or greater than 1, and wherein Rₐ, R_{b} and R_{c} are independently selected from H, alkyl, alkenyl and aryl.

p is preferably from 2-10, more preferably from 4-10, and most preferably p=8.

The different groups shown in the structure of formula (III) may be randomly dispersed along the polymer backbone; the structure does not imply a specific order or pattern of the different groups.

n, m and o are integers equal or greater than 1. The values of n, m and o are suitably sufficiently large that the pre-polymer has a weight average molecular weight as described above, e.g., from about 1,000 Daltons to about 1,000,000 Daltons.

According to the pre-polymer of general formula (III), some of the hydroxy groups on the backbone monomer units are activated with acrylate groups and some are reacting to present a negatively-charged phosphate groups. The preferred ratio of n:m:o will be determined by the preferred amounts of activated groups and negatively-charged functional groups.

In another embodiment of the invention the pre-polymer is of formula (IV):

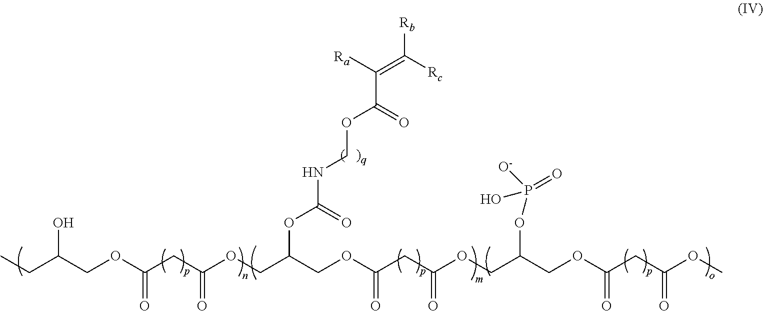

wherein p and q are integers between 1 and 20, wherein n, m and o are integers equal or greater than 1, and wherein Rₐ, R_{b} and R_{c} are independently selected from H, alkyl, alkenyl and aryl.

p is preferably from 2-10, more preferably from 4-10, and most preferably p=8.

q is preferably from 1-4, most preferably q is 2.

The different groups shown in the structure of formula (IV) may be randomly dispersed along the polymer backbone; the structure does not imply a specific order or pattern of the different groups.

n, m and o are integers equal or greater than 1. The values of n, m and o are suitably sufficiently large that the pre-polymer has a weight average molecular weight as described above, e.g., from about 1,000 Daltons to about 1,000,000 Daltons.

According to the pre-polymer of general formula (IV), some of the hydroxy groups on the backbone monomer units are activated with acrylate groups and some are reacting to present a negatively-charged phosphate groups. The preferred ratio of n:m:o will be determined by the preferred amounts of activated groups and negatively-charged functional groups.

A pre-polymer according to an embodiment of the invention and incorporating phosphate groups can be represented by the formula shown below:

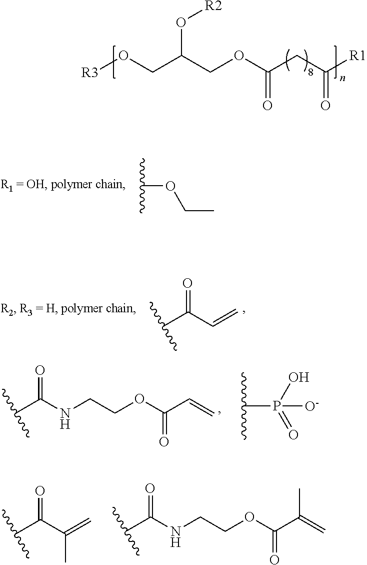

A pre-polymer according to an embodiment of the invention and incorporating carboxylate groups can be represented by the formula shown below:

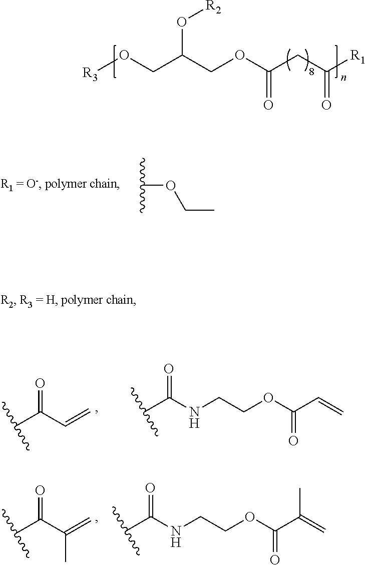

### Composition

The composition according to the present invention can be manufactured in the presence and/or mixed with a coloring agent. Preferred examples of coloring agents are the ones recommended by the US Food and Drug Administration (FDA) for use in medical devices, pharmaceutical products or cosmetics.

Similarly, the composition can further comprise stabilizers, for example MEHQ or N-Phenyl-2-naphthylamine (PBN).

The activated and functionalized pre-polymer of the composition can be further reacted with one or more additional materials to modify the crosslinks between the polymer chains. For example, prior to or during curing/crosslinking, one or more hydrogel or other oligomeric or monomeric or polymeric precursors (e.g., precursors that may be modified to contain acrylate groups), such as poly(ethylene glycol), dextran, chitosan, hyaluronic acid, alginate, other acrylate based precursors including, for example, acrylic acid, butyl acrylate, 2-ethylhexyl acrylate, methyl acrylate, ethyl acrylate, acrylonitrile, n-butanol, methyl methacrylate, acrylic anhydride, methacrylic anhydride and TMPTA, trimethylol propane trimethacrylate, pentaerythritol trimethacrylate, pentaerythritol tetramethacrylate, ethylene glycol dimethacrylate, dipentaerythritol penta acrylate, Bis-GMA (Bis phenol A glycidal methacrylate) and TEGDMA (tri-ethylene, glycol dimethacrylate), sucrose acrylate; other thiol based precursors (monomeric or polymeric); other epoxy based precursors; and combinations thereof, can be reacted with the pre-polymer.

The composition according to the present invention can be a surgical composition and is suitably used as a tissue sealant and/or adhesive. The composition suitably has flow characteristics such that it can be applied to the desired area through a syringe or catheter but is sufficiently viscous to remain in place at the site of application without being washed away by bodily fluids, such as water and/or blood.

Preferably, the viscosity of the composition is 500 to 100,000 cP, more preferably 1,000 to 50,000 cP, even more preferably 2,000 to 40,000 cP and most preferably 2,500 to 25,000 cP. Viscosity analysis is performed using a Brookfield DV-II+Pro viscosimeter with a 2.2 mL chamber and SC4-14 spindle, the speed during the analysis is varied from 5 to 80 rpm. The above-mentioned viscosity is present in the relevant temperature range for medical application i.e., room temperature up to 40° C., preferably 37° C.

The composition of the invention may be incubated in bodily fluids, such as blood, prior to administration and curing, without a substantial decrease in adhesive strength when cured.

The composition of the invention is suitably stable in bodily fluids, such as blood. More particularly, the composition of the invention suitably does not spontaneously crosslink in bodily fluids absent the presence of an intentionally applied stimulus, such as light, for example UV light, heat, or chemical initiator to initiate crosslinking.

The composition can be cured using a free radical initiated reaction, such as, for example, by photo-initiated polymerization, thermally-initiated polymerization, and redox initiated polymerization.

Preferably, the composition is irradiated with light, for example, ultraviolet (UV) light in the presence of a photoinitiator to facilitate the reaction. Examples of suitable photoinitiators include, but are not limited to: 2-dimethoxy-2-phenyl-acetophenone, 2-hydroxy-1[4-(hydroxyethoxy)phenyl]-2-methyl-1-propanone (Irgacure 2959), 1-hydroxycyclohexyl-1-phenyl ketone (Irgacure 184), 2-hydroxy-2-methyl-1-phenyl-1-propanone (Darocur 1173), 2-benzyl-2-(dimehylamino)-1[4-morpholinyl) phenyl]-1-butanone (Irgacure 369), methylbenzoylformate (Darocur MBF), oxy-phenyl-acetic acid-2[2-oxo-2-phenyl-acetoxy-ethoxy]-ethyl ester (Irgacure 754), 2-methyl-1[4-(methylthio)phenyl]-2-(4-morpholinyl)-1-propanone (Irgacure 907), diphenyl(2,4,6-trimethylbenzoyl)-phosphine oxide (Darocur TPO), phosphine oxide, phenyl bis(2,4,6-trimethyl benzoyl) (Irgacure 819), and combinations thereof.

Preferably, the composition is irradiated with visible light (typically blue light or green light) in the presence of a photoinitiator to facilitate the reaction. Examples of photoinitiators for visible light include, but are not limited to, diphenyl(2,4,6-trimethylbenzoyl)-phosphine oxide, eosin Y disodium salt, N-Vinyl-2-Pyrrolidone (NVP) and triethanolamine, and camphorquinone.

In applications of the composition involving in vivo photopolymerization and other medical applications, the use of cytocompatible photoinitiators is preferred and may be required by regulatory agencies. Photoinitiator Irgacure 2959 may be used, which causes minimal cytotoxicity (cell death) over a broad range of mammalian cell types and species.

In order for the photopolymerization to occur, the composition (and the substrate to which the composition is applied, if applicable) is preferably sufficiently transparent to the light.

In applications when the composition is cured in vivo, the temperature at which curing occurs is preferably controlled as not damage the tissue on which the composition has been applied. Preferably, the composition is not heated above 45° C. during irradiation, more preferably not above 37° C., and even more preferably not above 25° C.

In addition to photochemical crosslinking, the composition can be cured thermally, by Mitsunobu-type reaction, by redox-pair initiated polymerization for example benzoyl peroxide, N,N,-dimethyl-p-toluidine, ammonium persulfate, or tetramethylenediamine (TEMED), and by a Michael-type addition reaction using a bifunctional sulfhydryl compound.

In one embodiment, a redox composition (i.e., a composition that can be cured thermally by redox-pair initiated radical polymerization) may comprise 0.1 to 5 wt % of a reducing agent, e.g., 4-N,N Trimethylaniline, N,N-Bis(2-hydroxyethyl)-p-toluidine, N,N-Dimethylaniline, N,N-Diethylaniline, sodium p-toluenesulfonate or N-Methyl-N-(2-hydroxyethyl)-p-toluidine; 0 to 5 wt % of an oxygen inhibitor, e.g., 4-(Diphenylphosphino)styrene or triphenylphosphine; 0.005 to 0.5 wt % of a working time agent, e.g., Tempol or 4-methoxyphenol; and 0.1 to 10 wt % of an oxidant, e.g., ammonium persulfate, potassium persulfate or benzoyl peroxide. The reaction onset of the redox-pair initiated polymerization is affected by the absolute and relative amounts of the different reagents.

Upon polymerization, the activated and functionalized pre-polymer forms a crosslinked network with improved adhesive properties and exhibits significant adhesive strength even in the presence of blood and other bodily fluids. The cured polymer obtained after curing is preferably sufficiently elastic to resist movement of the underlying tissue, for example contractions of the heart and blood vessels. The adhesive can provide a seal, preventing the leakage of fluids or gas. The adhesive is preferably biodegradable and biocompatible, causing minimal inflammatory response. The adhesive is preferably elastomeric.

Biodegradability can be evaluated in vitro, such as in phosphate buffered saline (PBS) or in acidic or alkaline conditions. Biodegradability can also be evaluated in vivo, such as in an animal, for example mice, rats, dogs, pigs or humans. The rate of degradation can be evaluated by measuring the loss of mass of the polymer over time in vitro or in vivo.

The cured composition, alone or coated on a patch or tissue suitably exhibits a 90° pull off adhesive strength of at least 0.5 N/cm², preferably at least 1 N/cm² and even more preferably at least 2 N/cm², for example 1.5 N/cm² to 2 N/cm², but preferably greater than 5 N/cm², for example up to 6 N/cm² or 7 N/cm² or greater. Pull off adhesive strength refers to the adhesion value obtained by attaching an adhesive article or sample to wet tissue, such as epicardial surface of cardiac tissue or blood vessels immobilized on a flat substrate, such as a metallic stub. The 90° pull off adhesion test determines the greatest perpendicular force (in tension) that a surface area can bear before adhesive detachment (N. Lang et al., Sci. Transl. Med., 2014, 6, 218ra6).

According to a preferred embodiment, the composition of the invention is cured in light and in presence of a photo initiator and the cured composition exhibits a 90° pull off adhesive strength of at least 0.5 N/cm², preferably at least 1 N/cm², and even more preferably at least 2 N/cm², for example, 1.5 N/cm² to 2 N/cm², but preferably greater than 5 N/cm², for example up to 6 N/cm² or 7 N/cm² or greater.

The cured composition can desirably also exhibit a burst pressure of greater than 100 mmHg, preferably in the range of 400 mmHg to 600 mmHg or greater, for example 400 mmHg or 500 mmHg. Burst pressure or strength refers to the pressure value obtained to burst an explanted porcine carotid arterial vessel, which has an incision coated with the composition.

The composition of the present invention when cured in light and in the presence of a photo-initiator preferably has one or more of the following properties:
i) 90° pull off strength greater than 0.5 N/cm², preferably 2 to 7 N/cm² or greater; and
ii) burst performance of greater than 100 mmHg, preferably 200 to 300 mmHg or greater.

According to preferred embodiment, the composition of the invention is used as adhesive, i.e., it is able after curing to bind strongly to a surface or to bind one surface to another.

According to an alternative embodiment, the composition of the invention is used as sealant, i.e., it is able after curing to prevent leaking (e.g., of fluid or gas) by forming a barrier or filling a void volume.

Besides adhesion and sealing of wet biological tissue, the composition may adhere to and seal a variety of hydrophilic or hydrophobic substrates, natural or synthetic, including polyethylene terephthalate, expanded polyethylene terephthalate, polyester, polypropylene, silicones, polyurethanes, acrylics, fixed tissue (e.g., pericardium), ceramics or any combinations thereof.

### Method of Preparation

The method for preparing the composition of the present invention, comprises several required steps, which may accommodate several variations. According to a preferred embodiment, said method comprises the steps of:
i) polymerization of monomers to provide the polymeric backbone;
ii) activation of the polymeric backbone to provide the activated pre-polymer; and
iii) functionalization of the activated pre-polymer to provide the negatively-charged functional groups.

The monomers are preferably component A (polyol) and component B (polyacid) and are suitably added together in a molar ratio range of 0.5:1 to 1.5:1, preferably in the range of 0.9:1.1 to 1.1:0.9, and most preferably 1:1. Where component A is glycerol and component B is sebacic acid and added in a 1:1 molar ratio, there are three hydroxyl groups on glycerol for two carboxyl groups on the sebacic acid. Therefore, an extra hydroxyl group on glycerol is available for activation, as well as terminal carboxylic acid groups.

The conditions for step i) may include a temperature range of 100 to 140° C., preferably 120 to 130° C., an inert atmosphere, preferably comprising nitrogen, and under vacuum.

In a preferred embodiment, hydroxyl or carboxylic groups are present on the pre-polymer backbone obtained following step i).

The activation in step ii) is suitably achieved by acrylation of the pre-polymer backbone.

In a preferred embodiment, the activation is done through acrylation of the hydroxy or carboxyl groups. The activation of the carboxyl groups may result in the formation of anhydride that can be eliminated (totally or partially), for example using ethanol (see for example WO2016/202984).

One or more acrylates may be used as the acrylating agent. The acrylate may contain the following group: —C(═O)—CRₚ═CR_{q}Rᵣ, wherein Rₚ, R_{q}, Rᵣ are independently from one another, selected from the group consisting of H, alkyl, such as methyl or ethyl, aryl, such as phenyl, substituted alkyl, substituted aryl, carboxylic acid, ester, amide, amine, urethane, ether, and carbonyl. Preferably Rₚ is H. Most preferably, the acrylating agent is acryloyl chloride.

Step ii) can be carried out in the presence of one or more solvents or catalysts, examples including dichloromethane (DCM), ethyl acetate (EtOAc), dimethylaminopyridine (DMAP), and triethylamine (TEA) or any combination thereof.

Several purifications steps may be performed at this stage, preferably water washings steps, from 2 to 11 times, preferably 2 to 8 times, most preferably 8 times.

Alternatively, activation in step ii) can be acrylation using an isocyanate acrylate compound. A preferred isocyanate acrylate compound is 2-isocyanatoethyl(meth)acrylate.

For the functionalization step iii), in a preferred embodiment, hydroxy groups on the activated pre-polymer backbone are reacted to provide phosphate groups. A suitable reagent is phosphorus oxychloride (POCl₃). The reaction may take place at 0° C. under a nitrogen atmosphere. The resulting product may be hydrolyzed with water to obtain the negatively-charged phosphate group. Other suitable reagents include dialkyl chlorophosphonates, diphenylphosphinic chloride, phosphoric acid, orthophosphoric acid, phosphorus pentoxide, and diethyl chlorophosphite (see Illy, N. et al., Phosphorylation of bio-based compounds: The state of the art., Polym. Chem. 6, 6257-6291 (2015).), and may require specific reaction conditions to yield the activated functionalized pre-polymer.

For the functionalization step iii), in another embodiment, hydroxy groups on the activated pre-polymer backbone are reacted to provide sulphate groups. Sulphation methods are described by Al-Horani et al. in Tetrahedron 66,2907-2918 (2010).

For the functionalization step iii), in another embodiment, carboxylic acid groups on the polymeric backbone may be deprotonated to provide carboxylate groups. Deprotonation may be achieved by reaction with amines such as triethylamine or N,N-diisopropylethylamine.

According to one alternative embodiment, the functionalization step iii) yields a mixture of phosphate and carboxylate groups on the activated pre-polymer. It should be noted that at physiological pH, both phosphate and carboxylate negative charge can be simultaneously present.

According to another alternative embodiment, pre-polymer activation step ii) and functionalization step iii) may be inverted in the method of preparation of the activated and functionalized pre-polymer.

Different functionalization steps may be used in combination, e.g., the preferred amount of negatively-charged groups may be introduced by combining the introduction of phosphate or sulphate groups onto hydroxy groups with deprotonation of carboxylic acid groups.

At least one additive may be added to the composition obtained at step (iii). In a preferred embodiment, said additive is selected from the group consisting of photoinitiators, radical inhibitors, and dyes.

According to a preferred embodiment, the method further comprises one or more purification steps (iv) to ensure that solvents, by-products, impurities or un-reacted products are removed from the composition. These may be conducted throughout any reaction steps and more than one purification technique may be applied during the preparation of the composition.

In a preferred embodiment, such purification steps may include washes in aqueous media. Phase separation during water washings can be improved by the use of salts solubilized in the aqueous phase (e.g., from about 50 to about 500 g/L salt aqueous solution, preferably about 300 g/L salt, for example, sodium chloride, aqueous solution). According to a preferred embodiment, the washing uses salted water. Examples of salts include, but are not limited to, sodium chloride or potassium chloride.

According to a preferred embodiment, such purification steps may be conducted either by solvent evaporation or supercritical carbon dioxide extraction.

### Uses

### Tissue Adhesion and Sealing

The composition according to the invention may be used for adhering or sealing targeted surfaces including tissue, graft material, such as PTFE-based graft, or any combination thereof. The method for adhering or sealing targeted surfaces comprises applying the composition to the surface and curing the composition.

Unlike conventional tissue adhesives that spontaneously activate during application or in the presence of water, or adhesives that are hydrophilic and thus are subject to washout prior to curing, the composition according to the invention can be applied to wet substrates without activation or displacement. The composition can also be applied to dry substrates.

The composition may also be used for adhering tissue to the surface of a medical device. The composition can be used in medical devices, either as part or all of a device or to adhere a device to tissue. The method for adhering tissue to the surface of a medical device comprises applying the composition to the surface of the tissue and/or medical device and curing the composition. The composition can also be used to join tissue, including one or more tissue in vivo.

Surgical adhesives comprising the composition according to the invention can also be used for other applications. Examples of applications include to stop bleeding, for example, due to a wound or trauma, during surgery, such as after suturing a graft to a vessel or after vascular access in endovascular procedures. The adhesive does not need to be removed before the surgeon sutures the wound closed since it will degrade over time. Other types of wounds that can be treated include, but are not limited to, wounds that leak, or wounds that are hard to close or that fail to heal properly through normal physiologic mechanisms. The application can be performed both inside or outside the body, for human or veterinary use.

The composition according to the invention can also be fabricated into a biodegradable stent. The stent can increase the diameter of a blood vessel to increase flow through the vessel, but since the stent is biodegradable, the blood vessel can increase in diameter with a reduced risk of thrombosis or covering the stent with scar tissue, which can re-narrow the blood vessel. The composition can cover an outer surface of a stent to help adhere the stent to a vessel wall in a manner that is less damaging to the tissue than an uncovered stent or avoid its displacement inside the body. Similarly, the composition can cover the surface of any devices, which are in contact with tissue to provide a suitable interface that can be adhesive to tissue.

The composition according to the present invention can be used in a variety of other applications where an adhesive or sealant is required. These include, but are not limited to, air leaks following a lung resection; to reduce the time for surgical procedures; to seal dura; to ease laparoscopic procedures; as a degradable skin adhesive; as a hernia matrix to prevent or to reduce the need for stables or tacks; to prevent blood loss; to manipulate organs or tissues during surgical procedures; to secure corneal transplants in place; to patch a heart to deliver drugs and/or to reduce dilation of the heart after myocardial infarction; to attach another material to a tissue; to augment sutures or staples; to distribute forces across tissue; to prevent leaks; as a barrier membrane on the skin to prevent evaporation of water from burnt skin; as a patch for delivery of anti-scar or antimicrobial medication; to attached devices to tissue; to attach devices to mucus membrane as a tape to secure devices within an oral cavity, such as to hold dentures and oral appliances; as a tape to anchor soft tissue to bone; and, preventing the formation of holes in tissue, enhancing/augmenting mechanical properties of tissues, etc.

### Delivery of Bioactive Molecules

The composition according to the invention may also contain one or more pharmaceutical, therapeutic, prophylactic, and/or diagnostic agents that are released during the time period that the material functions as a sealant/adhesive. The agent may be a small molecule agent, for example, having molecular weight less than 2000, 1500, 1000, 750, or 500 Da, a biomolecule, for example, peptide, protein, enzyme, nucleic acid, polysaccharide, growth factors, cell adhesion sequences such as RGD sequences or integrins, extracellular matrix components, or combinations thereof. Exemplary classes of small molecule agents include, but are not limited to, anti-inflammatories, immunosuppressive molecules (e.g. tacrolimus, cyclosporin), analgesics, antimicrobial agents, antibiotics, and combinations thereof. Exemplary growth factors include, without limitation, TGFβ, acidic fibroblast growth factor, basic fibroblast growth factor, epidermal growth factor, IGF-I and II, vascular endothelial-derived growth factor, bone morphogenetic proteins, platelet-derived growth factor, heparin-binding growth factor, hematopoietic growth factor, peptide growth factor, or nucleic acids. Exemplary extracellular matrix components include, but are not limited to, collagen, fibronectin, laminin, elastin, and combinations thereof. Proteoglycans and glycosaminoglycans can also be covalently or non-covalently associate with the composition of the present invention.

### Tissue Support

The composition according to the invention can be used to create tissue supports by forming shaped articles within the body to serve a mechanical function. The shaped articles may be produced by a variety of fabrication techniques know in the art, including 3D printing. Such articles may exert functions, such as holding two tissues together or positioning the tissue in a specific position inside or outside the body.

The tissue can be coated with a layer of the materials, for example, the lumen of a tissue, such as a blood vessel to prevent restenosis, reclosure or vasospasm after vascular intervention.

The composition may also contain one or more types of cells, such as connective tissue cells, organ cells, muscle cells, nerve cells, and combinations thereof. Optionally, the material is seeded with one or more of tenocytes, fibroblasts, ligament cells, endothelial cells, lung cells, epithelial cells, smooth muscle cells, cardiac muscle cells, skeletal muscle cells, islet cells, nerve cells, hepatocytes, kidney cells, bladder cells, urothelial cells, chondrocytes, and bone-forming cells. The combination of cells with the material may be used to support tissue repair and regeneration.

### Anti-Adhesion Barriers

The composition according to the invention herein described can be applied to reduce or prevent the formation of adhesions after surgical procedures. For example, the composition can be applied to prevent adhesion of brain tissue to the skull after brain surgery or implantation of devices to prevent peritoneal adhesion.

### Other Applications

The compositions can also be used to coat tools, such as surgical instruments, for example, forceps or retractors, to enhance the ability of the tools to manipulate objects. The compositions can also be used in industrial applications where it is useful to have a degradable adhesive that is biocompatible, for example, to reduce potential toxicity of the degradation products, such as marine applications, for example, in underwater use or attaching to the surface of boats. The compositions can be also used to produce shaped objects by a variety of techniques known in the art, including 3D printing. The shaped object may have micro or nanoscale resolution.

The present invention will now be illustrated, but in no way limited, by reference to the following examples.

### EXAMPLES

### Adhesion Performances

The adhesive performances in the following examples were tested by pull-off adhesion according to the following pull off method. Pull-off adhesion testing (at 90°) was performed on an Instron with fresh porcine epicardial tissue. The tissue was kept in phosphate-buffered saline to assure that it remained wet during testing. Unless specified, a poly glycerol sebacate urethane (PGSU) patch was used for testing and was about 200 mm thick and 6 mm in diameter. A thin layer of pre-polymer, with a thickness of about 200 μm, was applied to the patch material before adhesion testing. During the curing process, a compressive force of 3 N was applied to the sample composition coated patch with a non-adhesive material (borosilicate glass rod 9 mm in height) connected to the UV light guide (Lumen Dynamics Group Inc) with standard adhesive tape around both the glass rod and the light guide. The interposition of the borosilicate glass rod facilitates the release of the curing system from the patch without disturbing the patch/adhesive-tissue interface. The pull-off procedure involved grip separation at a rate of 8 mm/min, causing uniform patch detachment from the tissue surface. Adhesion force was recorded as the maximum force observed before adhesive failure, when a sharp decrease in the measured stress was observed.

### Example 1: Synthesis of Activated and Phosphorylated PGS

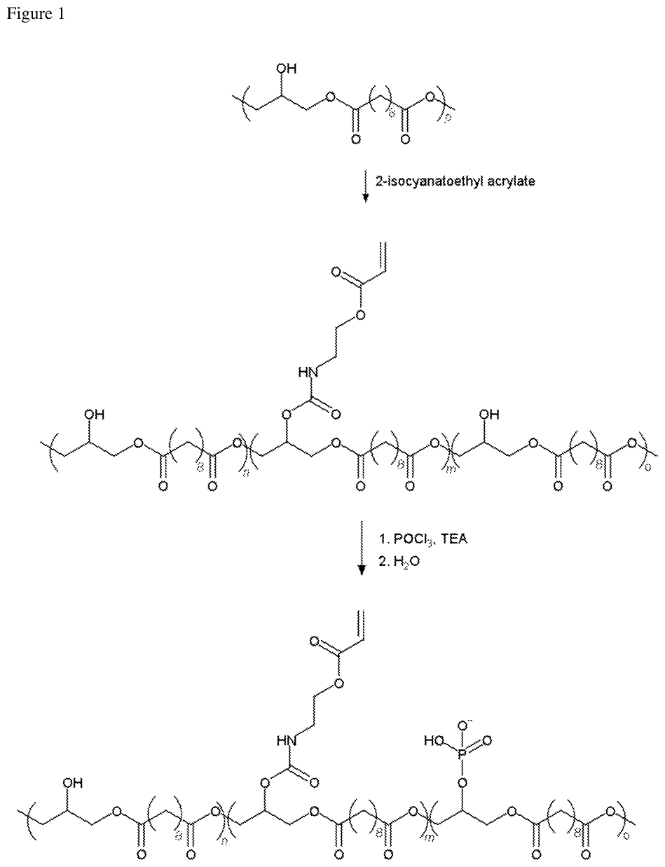

The synthetic steps used in this Example are shown in FIG. 1.

### (i) Synthesis of Poly(Glycerol Sebacate) (PGS)

1. Equimolar amounts of glycerol and sebacic acid were weighed.
2. The reaction mixture temperature set between 120 and 130° C. until the monomers were completely melted.
3. Upon melting of the reagents, the bath or reaction temperature was reduced to the target value of 120° C. and stirring started.
4. The air inside the flask was replaced with nitrogen using three vacuum/purging cycles.
5. The reaction was followed for 8 hours.
6. The nitrogen supply was then removed, and the pressure reduced using a vacuum pump set to a target of 15 mBars.

The reaction was followed until the targeted Mw (about 3,000 Da) and polydispersity (<3) were achieved. The glycerol : sebacic acid molar ratio targeted was 1:1, as confirmed by nuclear magnetic resonance (NMR).

### (ii) Activation (Acrylation) of PGS

The following procedure was used to activate hydroxide groups on the PGS backbone:

40 g of PGS were added to 50 ml of dichloromethane (DCM). 6.12 g of 2-isocyanatoethyl acrylate (0.3 equivalents per monomer of polyol) were added. The mixture was stirred at 40° C. for 32 hours. The DA of the product was measured to be 0.3 mol/mol of polyacid.

(iii) Functionalization

The acrylated PGS obtained from step (ii) was reacted with phosphorus oxychloride (0.2 equivalents per monomer of polyol) at 0° C. under a nitrogen atmosphere. The resulting product was hydrolyzed by water to obtain the phosphorylated product.

The resulting material was concentrated under reduced pressure and was purified by scCO₂ extraction. The DA of the product was 0.25 mol/mol of polyacid. The degree of phosphorylation (i.e., the amount of negatively-charged groups) was 0.2 mol/mol of polyacid.

The adhesive strength of several samples of the material was measured using heart pull-off testing as described above and in N. Lang et al., Sci. Transl. Med., 2014, 6, 218ra6. The adhesion value was 7.7±3.2 N/cm².

### Example 2A: Synthesis of Activated PGS and Functionalization with Carboxylate Groups

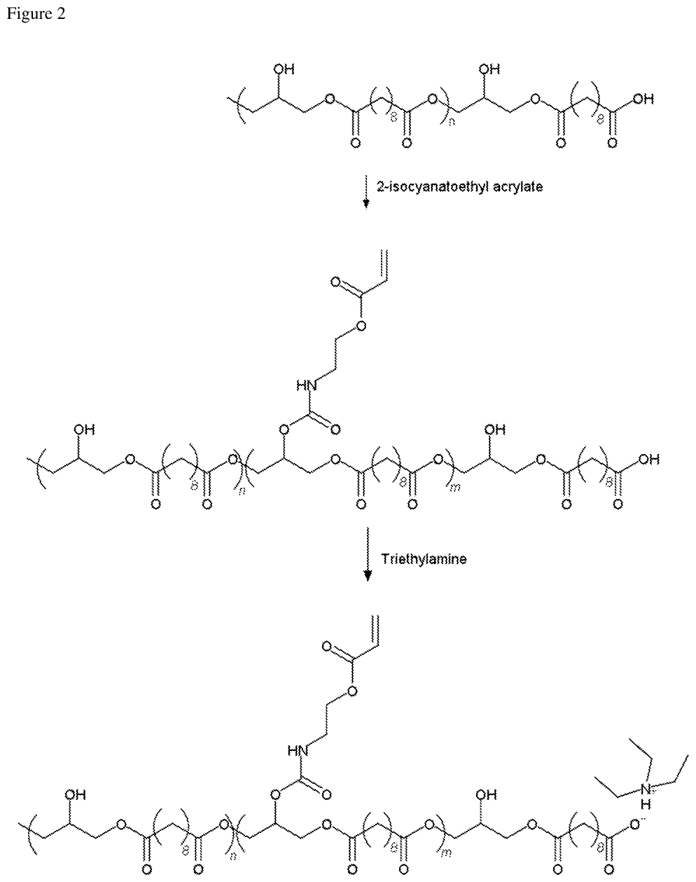

The synthetic steps used in this Example are shown in FIG. 2.

Steps (i) was carried out as in Example 1 above.

499.98 g of PGS (previously melted at 80° C.) were weighed in a 2 L flask, 1.110 L of EtOAc were added. 101.57 mL of 2-isocyanatoethyl acrylate were added to the mixture. The mixture was stirred at 70° C. for 10 h.

The acrylated PGS obtained was purified by scCO₂ extraction. It was then reacted with a tertiary amine (triethylamine, 1.4 mmol/g of polymer), in excess compared to the amount of carboxylic acids in the polymer backbone. The resulting polymer was analyzed by ¹H NMR. The DA of the product was 0.43 mol/mol of polyacid. The amount of carboxylate groups (i.e., the amount of negatively-charged groups) was 0.36 mol/mol of polyacid.

The adhesive strength of several samples of the material was measured using heart pull-off testing. The adhesion value was 5.0±2.2 N/cm².

### Example 2B: Synthesis of Activated PGS and Functionalization with Carboxylate Groups

The method of Example 2A was repeated, except that N,N-diisopropylethylamine (1.4 mmol/g of polymer) was used instead of triethylamine. The resulting polymer was analyzed by ¹H NMR. The DA of the product was 0.45 mol/mol of polyacid. The amount of carboxylate groups (i.e., the amount of negatively-charged groups) was 0.16 mol/mol of polyacid.

The adhesive strength of several samples of the material was measured using heart pull-off testing. The adhesion value was 4.9±1.9 N/cm².

### Example 3: Activation (Acrylation) and Functionalization of PGS with Carboxylate Groups

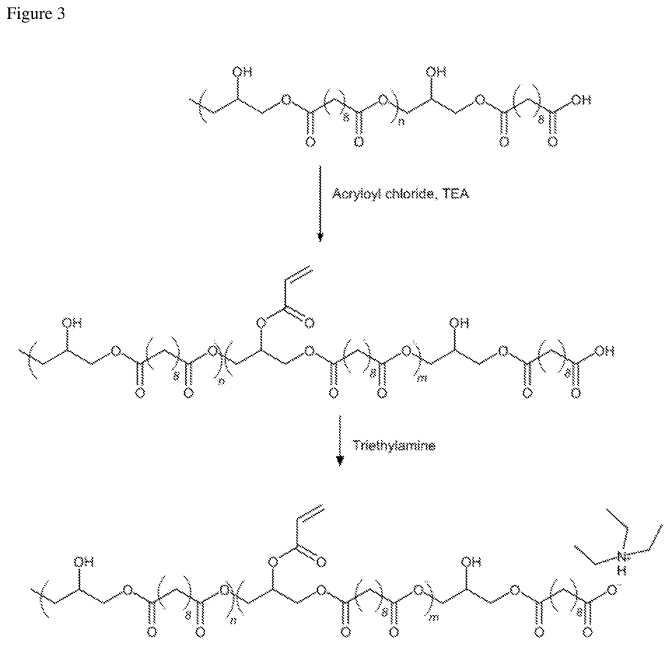

The synthetic steps used in this Example are shown in FIG. 3.

The following procedure was used to activate hydroxy groups on the PGS backbone. The PGS was reacted with acryloyl chloride (˜0.37 g of acryloyl chloride (AcCl) per 1 g of PGS) in 10% (w/v) dichloromethane (DCM) and triethylamine (˜0.4 g of triethylamine (TEA) per 1 g of PGS). Ethanol capping of the acrylated PGS was achieved by reaction with ethanol, overnight, at a temperature in the range of between 30 and 50° C. The resulting pre-polymer is purified by water washings, preferably 8 times, and was distilled to yield pre-polymer poly(glycerol sebacate)acrylate, PGSA.

PGSA (500 mg) was reacted with a triethylamine (0.7 mmol). The resulting polymer was analyzed by ¹H NMR. The DA of the product was 0.45 mol/mol of polyacid. The amount of carboxylate groups (i.e., the amount of negatively-charged groups) was 0.16 mol/mol of polyacid.

The adhesive strength of several samples of the material was measured using heart pull-off testing. The adhesion value was 7.4±4.5 N/cm².

### Example 4: Activation (Acrylation) and Functionalization of PGS with Carboxylate Groups

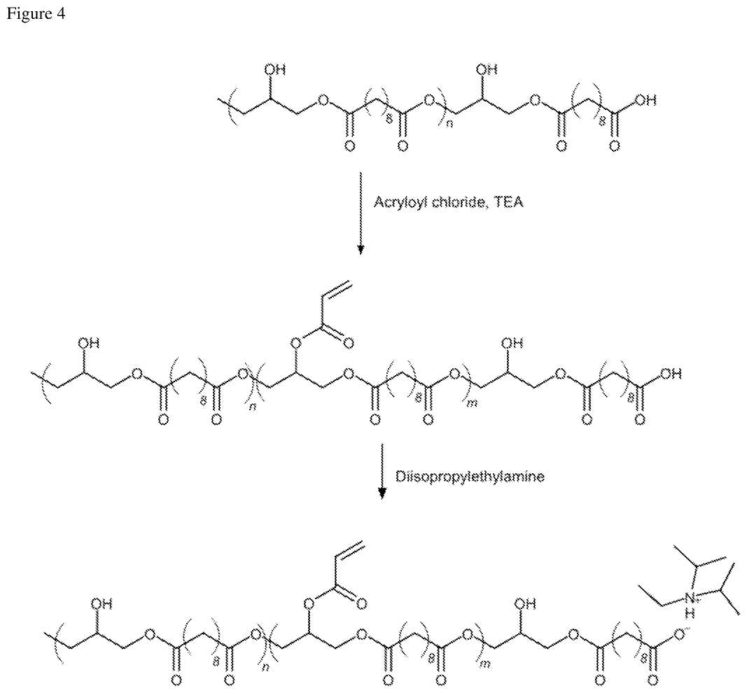

The synthetic steps used in this Example are shown in FIG. 4.

The following procedure was used to activate hydroxy groups on the PGS backbone. The PGS was reacted with acryloyl chloride (˜0.37 g of acryloyl chloride (AcCl) per 1 g of PGS) in 10% (w/v) dichloromethane (DCM) and triethylamine (˜0.4 g of triethylamine (TEA) per 1 g of PGS). Ethanol capping of the acrylated PGS was achieved by reaction with ethanol, overnight, at a temperature in the range of between 30 and 50° C. The resulting pre-polymer is purified by water washings, preferably 8 times, and was distilled to yield pre-polymer poly(glycerol sebacate)acrylate, PGSA.

PGSA (500 mg) was reacted with a diisopropylamine (0.7 mmol). The resulting polymer was analyzed by ¹H NMR. The DA of the product was 0.45 mol/mol of polyacid. The amount of carboxylate groups (i.e., the amount of negatively-charged groups) was 0.16 mol/mol of polyacid.

The adhesive strength of several samples of the material was measured using heart pull-off testing. The adhesion value was 4.9±1.9 N/cm².

1. A composition comprising:
a pre-polymer having activated groups and negatively-charged functional groups on a polymeric backbone, wherein the proportion of negatively-charged functional groups compared to the number of monomer units in the backbone is at least 0.05 mol/mol of monomer unit.

2. The composition according to claim 1, wherein the proportion of negatively-charged functional groups compared to the number of monomer units in the backbone is at least 0.1 mol/mol of monomer unit, preferably from 0.2 mol/mol of monomer unit.

3. The composition according to claim 1 or claim 2, wherein the negatively-charged functional groups are chosen from phosphate, sulphate and carboxylate groups.

4. The composition according to claim 3, wherein the negatively-charged functional groups are chosen from phosphate groups and sulphate groups.

5. The composition according to claim 4, wherein the negatively-charged functional groups are phosphate groups.

6. The composition according to any preceding claim, wherein the activated groups are acrylate groups or vinyl groups, preferably acrylate groups.

7. The composition according to any preceding claim, wherein the proportion of activated groups compared to the number of monomer units in the backbone is from 0.05 to 0.4 mol/mol of polyacid or polyol, preferably from 0.09 to 0.25 mol/mol of monomer unit.

8. The composition according to any preceding claim, wherein the polymeric backbone of the pre-polymer is of the formula (—A—B—)ₙ, wherein A is derived from a substituted or unsubstituted polyol and B is derived from a substituted or unsubstituted polyacid, preferably diacid or a triacid, and n is greater than 1.

9. The composition according to claim 8, wherein the polyol is a triol, preferably glycerol or trimethylolpropane ethoxylate, and wherein B is a diacid selected from the group consisting of glutaric acid, adipic acid, pimelic acid, sebacic acid and azelaic acid, preferably sebacic acid.

10. The composition according to claim 8, wherein the polyol is a diol, preferably octanediol, and wherein B is a triacid, preferably citric acid.

11. The composition according to any preceding claim, wherein the pre-polymer is of formula (III) or (IV): 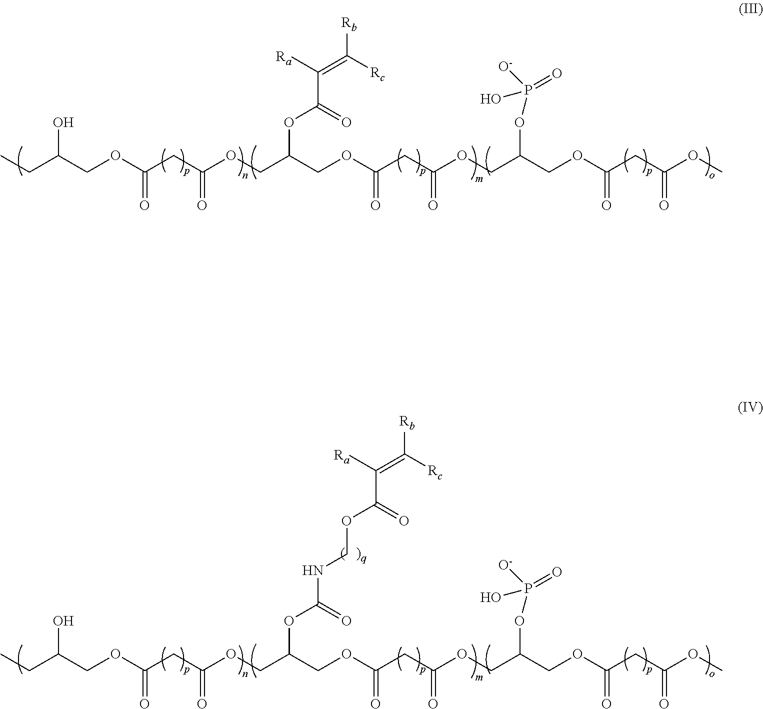
wherein p and q are integers between 1 and 20, wherein n, m and o are integers equal or greater than 1, and wherein Rₐ, R_{b} and R_{c} are independently selected from H, alkyl, alkenyl and aryl.

12. A composition according to any preceding claim further comprising an initiator.

13. A method for preparing a composition according to any preceding claim, comprising steps of:
i) polymerization of monomers to provide the polymeric backbone;
ii) activation of the polymeric backbone to provide the activated pre-polymer; and
iii) functionalization of the activated pre-polymer to provide the negatively-charged functional groups.

14. A method according to claim 13, wherein the monomers that provide the polymeric backbone include a polyol, preferably a triol such as glycerol, and a diacid or a triacid, preferably sebacic acid.

15. A method according to claim 13 or claim 14, wherein the activation in step (ii) is achieved by acrylation of the hydroxide groups to give acrylate groups.

16. A method according to any one of claims 13 to 15, wherein the functionalization in step (iii) is achieved by reaction with phosphorus oxychloride.

17. A method of curing a composition according any one of claims 1-12 or a composition obtainable by a method according to any one of claims 13-16, comprising a step of curing the composition with as stimulus, preferably with light in the presence of a photo-initiator.

18. A composition according to any one of claims 1-12 or a composition obtainable by a method according to any one of claims 13-16, for use in a method of adhering or sealing tissue, or for adhering tissue to the surface of a medical device.

19. A cured composition obtainable by the method of claim 17.

20. Use of a composition according to any one of claims 1-12 or a composition obtainable by a method according to any one of claims 13-16, in a method of adhering or sealing tissue, or for adhering medical device to the surface of a tissue.
